(19) United States
(12) Patent Application Publication
(10) Pub. No.: US 2023/0183440 A1
(43) Pub. Date: Jun. 15, 2023

(54) CHEMOENZYMATIC DEGRADATION OF EPOXY RESINS

(71) Applicants: Solvay SA, Brussels (BE); RHODIA POLIAMIDA E ESPECIALIDADES S.A., São Paulo (BR)

(72) Inventors: Victor Ferrao, Sao Paulo (BR); Stephane Streiff, Shanghai (CN); Priscila Maziero, Sao Paulo (BR); Fan Jiang, Yunnan (CN); Tao Lin, Shanghai (CN)

(73) Assignees: Solvay SA, Brussels (BE); RHODIA POLIAMIDA E ESPECIALIDADES S.A., São Paulo (BR)

(21) Appl. No.: 17/926,497

(22) PCT Filed: May 29, 2020
(86) PCT No.: PCT/CN2020/093274

§ 371 Date: Nov. 18, 2022

(51) Int. Cl. C08J 11/10; C08J 11/08; C08G 59/24; C08G 59/50; C12N 9/10

(52) U.S. Cl. CPC C08J 11/105; C08J 11/08; C08G 59/245; C08G 59/5073; C12N 9/1088; C08J 2363/00

(57) ABSTRACT

Provided is a method for degrading epoxy resins by enzymatic route, in particular by adding an epoxy resin in a solvent followed by treatment with a glutathione S-transferase. A method for recycling a composite material comprising epoxy resin as well as the use of glutathione 5-transferase and eventually para-hydroxybenzoate hydroxylase for degrading epoxy resins are also provided.

### FIELD OF INVENTION

The invention deals with a method for degrading epoxy resins by enzymatic route, in particular by adding an epoxy resin in a solvent followed by treatment with a glutathione S-transferase. The invention also deals with a method for recycling a composite material comprising epoxy resin as well as the use of glutathione S-transferase and eventually para-hydroxybenzoate hydroxylase for degrading epoxy resins.

### BACKGROUND OF THE INVENTION

Composite material comprising carbon-fiber and epoxy resins are widely used notably in aeronautics, aerospace and automotive industries.

The growing development of products comprising such composite material implies also growing amount of waste at the end of these product's life. Disposal of composite material waste should be addressed to protect the environment.

Degradation of epoxy resin comprised in the composite material can be envisioned. It could notably allow recovery of carbon fibers from composite material.

Different kinds of degradations have already been developed such as thermal degradation (pyrolytic decomposition of epoxy resin), chemical degradation (degradation of epoxy resin by acids or supercritical fluids) or mechanical degradation (shredding, crushing and grinding of the composite material). However, these techniques have several drawbacks. They tend to either lower the quality of the carbon fibers, or they are very energy consuming and imply handling toxic substances.

Enzymatic degradation implies generally mild and safe conditions which have less impact on the environment.

Enzymatic degradation of polymers has been essentially evaluated on lignin, polyesters, polyamides or polyurethanes (US 2016/0280881, US 2009/0162337, WO 99/29885, U.S. Pat. No. 6,255,451), but not on epoxy resins.

Eliaz et al. (Materials 2018, 11, 2123) have evaluated microbial degradation of epoxy resins. Two bacterial species were identified as being potentially able to degrade epoxy resins: Rhodococcus rhodochrous and Ochrobactrum anthropi.

Moeser et al. (Advanced Materials Research 2014, 1018, 131-136) report also evaluation of degradation of uncured epoxy resins by microorganisms. However, the results are not clear.

These documents are not concluding about the effectiveness of the degradation and about the enzymes that could be used for it.

The applicant found that epoxy resins could be degraded by enzymatic route effectively.

### SUMMARY OF THE INVENTION

The present invention deals with a method for degrading an epoxy resin comprising the following successive steps:

a) adding in a solvent an epoxy resin R based on:
at least one aromatic compound R1 bearing at least two epoxide groups per molecule and comprising at least one aromatic ring bearing at least one glycidyloxy group, and
at least one curing agent R2,

to obtain mixture MA,

b) treating enzymatically the mixture MA obtained after step a), the treatment comprising a step of contacting the mixture MA with a glutathione S-transferase to obtain a mixture MB.

Preferably, the method of the invention further comprises a step c) performed after step b) or concomitantly with step b), step c) consisting in an enzymatic treatment which comprises a step of contacting mixture MA or mixture MB with a para-hydroxybenzoate hydroxylase to obtain a mixture MC.

In particular, enzymatic treatment consisting in step b) or step b) and step c) is followed by a successive step d) which consists in treating chemically mixture MB or mixture MC, the chemical treatment comprising a step of contacting mixture MB or mixture MC with an aqueous solution of a strong Bronsted base to obtain a mixture MD. More particularly, the aqueous solution of a strong Bronsted base in step d) is a sodium hydroxide aqueous solution.

Advantageously, step a) comprises swelling of the epoxy resin R in the solvent. More advantageously, in step a) the solvent is chosen among the group consisting in an aqueous solution of disodium phosphate, an aqueous solution of phosphoric acid and phenol, more preferably the solvent is an aqueous solution of disodium phosphate.

In particular, in step b) the glutathione S-transferase is issued from a N. aromaticivorans strain.

Preferably, in step c) the para-hydroxybenzoate hydroxylase is a mutated enzyme, preferentially is a mutated enzyme presenting at least two point mutations L199V or L200V and Y385F.

Advantageously, the aromatic compound R1 corresponds to a diglycidyl ether of bisphenol, in particular a bisphenol A diglycidyl ether.

In particular, the curing agent R2 is an amine or an imidazole derivative.

In particular, mixture MD obtained after step d) is then further submitted to steps b) to d) as defined above. Alternatively, mixture MD obtained after step d) is further submitted to steps a) to d) as defined above.

The present invention also relates to the use of a glutathione S-transferase, in particular a glutathione S-transferase issued from a N. aromaticivorans strain, for degrading an epoxy resin R based on:
at least one aromatic compound R1 bearing at least two epoxide groups per molecule and comprising at least one aromatic ring bearing at least one glycidyloxy group, and
at least one curing agent R2.

In particular, a para-hydroxybenzoate hydroxylase is further used for degrading the epoxy resin R, preferably a mutated para-hydroxybenzoate hydroxylase presenting at least two point mutations: L199V or L200V and Y385F, for degrading an epoxy resin R based on:
at least one aromatic compound R1 bearing at least two epoxide groups per molecule and comprising at least one aromatic ring bearing at least one glycidyloxy group, and
at least one curing agent R2.

The present invention deals with a method for recycling a composite material CM comprising the following steps:

a′) providing a composite material comprising an epoxy resin R based on:
at least one aromatic compound R1 bearing at least two epoxide groups per molecule and comprising at least one aromatic ring bearing at least one glycidyloxy group, and
at least one curing agent R2,

b′) treating the composite material CM obtained after step a′), the treatment comprising a step of degrading the epoxy resin R with the method for degrading an epoxy resin according to the invention, in which case the epoxy resin R in step a) is the composite material CM,

c′) obtaining after step b′) the composite material with a reduced content in epoxy resin.

1. Method for Degrading an Epoxy Resin

The method according to the invention comprises at least two steps a) and b) described as follows.

1.1 Step a)

Step a) comprises adding in a solvent an epoxy resin R based on:
at least one aromatic compound R1 bearing at least two epoxide groups per molecule and comprising at least one aromatic ring bearing at least one glycidyloxy group, and
at least one curing agent R2 to obtain mixture MA.

Step a) allows swelling of the epoxy resin R in the solvent to which it is added. In particular, it can also allow decrosslinking of the epoxy resin R. More particularly, when the solvent is an aqueous solution comprising phosphate, step a) allows phosphorylation of the epoxy resin or the partially decrosslinked epoxy resin.

In the present invention, a nonpolar solvent is a solvent with a dielectric constant of less than 15.

In the present invention, a polar solvent is a solvent with a dielectric constant of 15 or more than 15.

In the present invention, a polar protic solvent is a solvent with a dielectric constant of 15 or more than 15 and containing a labile proton.

In the present invention, a polar aprotic solvent is a solvent with a dielectric constant of 15 or more than 15 and lacking a labile proton.

The solvent can be a nonpolar solvent chosen from pentane, cyclopentane, hexane, cyclohexane, heptane, petroleum ether, toluene, xylene, chloroform, dichloromethane and mixtures thereof, in particular it is hexane.

The solvent can be an aprotic polar solvent chosen from dimethyl sulfoxide (DMSO), dimethylformamide (DMF), dimethylacetamide (DMA), tetrahydrofuran (THF), N-methyl-2-pyrrolidone (NMP), dimethoxy ethane (DME), methyl tert-butyl ether (MTBE), nitromethane, 1,4-dioxane, acetonitrile, acetone, ethyl acetate, diethyl ether, acetic acid, formic acid and mixtures thereof, in particular it is chosen from DMSO and DMF.

The solvent can be a protic polar solvent chosen from an alcohol, an aqueous solution and mixtures thereof.

In the present invention, an alcohol is an organic compound carrying at least one hydroxyl group to a carbon atom. Preferably it is liquid at a temperature comprised between 55° C. and 95° C. In particular, it can be methanol, ethanol, n-propanol, isopropanol, butanol and regioisomers thereof, phenol, pentanol and regioisomers thereof, 1-hexanol, 2,2,2-trifluoroethanol (TFE), hexafluoropropan-2-ol (HFIP).

The solvent to which the epoxy resin R is added can be a mixture of nonpolar solvents and aprotic polar solvents as described above.

The solvent to which the epoxy resin R is added can be a mixture of nonpolar solvents and protic polar solvents as described above.

The solvent to which the epoxy resin R is added can be a mixture of aprotic polar solvents and protic polar solvents as described above.

Preferably, the solvent to which the epoxy resin R is added is chosen from a polar protic solvent, a polar aprotic solvent and mixtures thereof as described above, more particularly it is a polar protic solvent as described above.

Advantageously, the solvent is chosen among the group consisting of an alcohol, an aqueous solution and a mixture thereof. More advantageously the aqueous solution can comprise one or more Bronsted acid, such as H₃PO₄, H₂SO₄, HCl, HBr or HNO₃. Alternatively, the aqueous solution can comprise one or more Bronsted base, such as disodium phosphate, ammonia, NaOH, KOH, Na₂CO₃ or K₂CO₃.

In particular, the solvent to which the epoxy resin R is added is chosen among the group consisting in phenol, an aqueous solution of phosphoric acid, an aqueous solution of disodium phosphate and a combination thereof. More particularly, the solvent of step a) is an aqueous solution of disodium phosphate. Even more particularly, the aqueous solution of disodium phosphate used in step a) is of a concentration from 120 to 1000 g/mL of disodium phosphate, preferably 120 to 500 g/L. Notably, disodium phosphate is used in its form of disodium phosphate dodecahydrate.

Advantageously, the weight ratio disodium phosphate/epoxy resin is comprised between 5 and 150 g/g.

The mixture of epoxy resin R and solvent of step a) can be heated at a temperature comprised between 20° C. and 95° C., preferably between 40° C. and 95° C., more preferably between 55° C. and 95° C.

Step a) is preferably performed under stirring.

Step a) can lasts from 72 hours to 4 days.

Advantageously, step a) comprises the following steps:
a1) adding epoxy resin R to the solvent,
a2) stirring, then
a3) optionally partially evaporating under reduced pressure, and then
a4) optionally diluting with water.

The four steps a1), a2), a3) and a4) are successive. Steps a2), a3) and optionally a4) can be repeated one time or more than one time, in particular from one time to three times.

In particular, in step a2) the mixture of epoxy resin R in the solvent is stirred for from 72 hrs to 1920 hrs.

In particular, in step a2) the mixture of epoxy resin R in the solvent is stirred at a temperature comprised between 20° C. and 95° C., preferably between 40° C. and 95° C., more preferably between 55° C. and 95° C.

Preferably, cyclodextrin is further added to the mixture with epoxy resin in the solvent.

In the present invention, a cyclodextrin is a cyclic oligosaccharide, consisting of a macrocyclic ring of α-D-glucopyranose subunits joined by α-1,4 glycosidic bonds. It is in particular composed of five or more α-D-glucopyranose units linked by 1,4 glycosidic bonds. Cyclodextrin α comprises 6 α-D-glucopyranose subunits. Cyclodextrin β comprises 7 α-D-glucopyranose subunits. Cyclodextrin γ comprises 8 α-D-glucopyranose subunits.

Preferably, the cyclodextrin used in step a) comprises a number of D-glucopyranose subunits ranging from six to eight units in a ring.

More preferably the cyclodextrin is chosen among the group consisting in cyclodextrin α, cyclodextrin β and cyclodextrin γ, even more preferably the cyclodextrin is cyclodextrin β.

Advantageously, the weight ratio cyclodextrin/epoxy resin is comprised between 2.5 and 50 g/g.

Advantageously step a) comprises the following steps:
a5) adding cyclodextrin to the mixture of epoxy resin R and solvent,
a6) heating the resulting mixture at a temperature comprised between 20° C. and 95° C.

Steps a5) and a6) are preferably performed under agitation.

In particular, the cyclodextrin is added after step a4). More particularly, steps a5) and a6) follow step a4).

Preferably, in step a6), the mixture of epoxy resin, cyclodextrin and solvent is stirred for 24 hours to several weeks, more preferably for 36 hours to 72 hours.

The mixture obtained after step a) is called mixture MA.

1.2 Step b)

Step b) comprises treating enzymatically the mixture MA obtained after step a), the treatment comprising a step of contacting the mixture MA with a glutathione 5-transferase to obtain a mixture MB.

In the sense of the invention, a “glutathione S-tranferase” (GST) is an enzyme having the ability to catalyze the conjugation of the reduced form of glutathione (GSH) with different substrates.

In the normalized nomenclature, these enzymes are classified as members of EC 2.5.1.18.

In particular, the GST enzyme is able to transfer the glutathione group on the structure of the resin, as shown in the equation below (or figure X):

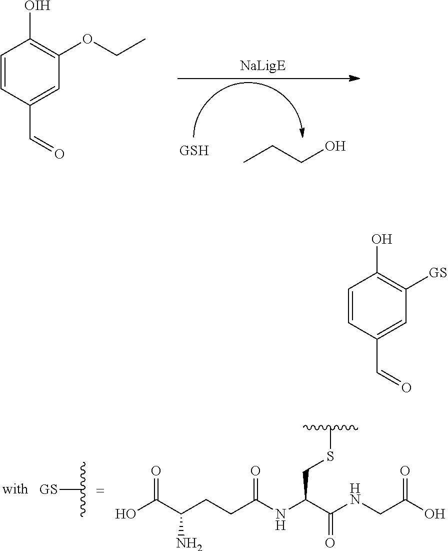

Indeed, glutathione or (2S)-2-Amino-4-{[(1R)-1-[(carboxymethyl)carbamoyl]-2-sulfanylethyl]carbamoyl}butanoic acid comprises one thiol group. The glutathione group corresponds to the molecule of glutathione linked through its sulfur to the structure of the resin.

The glutathione S-transferases (GSTs) used in the process of the invention may be of eukaryotic or prokaryotic origin, preferentially is of prokaryotic origin.

Among the superfamily of GSTs, a subclass of stereospecific glutathione S-transferases called β-etherases are of particular interest for the process of the invention.

In particular, the enzymes LigE, LigP, and LigF issued from Sphingobium sp. and their homologs may be used. Among the homologs easily identified by the man skilled in the art, one can cite the enzymes LigE-NS(=NsLigE) and LigF-NS from Novosphingobium sp. PP1Y, LigE-NA (=NaLigE), LigF-NA (=NaLigF1) and NaLigF2 from Novosphingobium aromaticivorans DSM12444.

In a specific implementation of the process, the glutathione S-transferase used in step b) is a β-etherase. In particular, the GST used in step b) is an enzyme LigE, LigP or LigF issued from Sphingobium sp, or any homolog of these enzymes, for example LigD, LigL, LigN, LigO or LigG. Preferentially, the GST used in step b) is a LigE homolog.

In a specific implementation of the process of the invention, in step b), the glutathione S-transferase is issued from Novosphingobium aromaticivorans (N. aromaticivorans) strain. The glutathione S-transferase may be in particular a LigE homolog issued from Novosphingobium aromaticivorans strain, i.e. LigE-NA.

In particular, the glutathione S-transferase can be immobilized on a suitable solid support.

Advantageously, the glutathione S-transferase is added to the mixture MA in the form of a broth of lysed bacteria expressing glutathione S-transferase. The bacteria are as described above. The broth of lysed bacteria is obtained by cultivation of bacteria as described above followed by centrifugation to obtain cell pellets, then cell pellets are mixed with phosphate buffer (0.1M pH=7.0) in a proportion of 1 g (of pellets)/10 ml (of buffer) and bacteria are lysed by sonication.

The broth obtained from the bacteria lysis contains the glutathione S-transferase. At least 1 vol. % of this solution can be used, preferably from 1 vol. % to 20 vol. % can be used.

In particular, in step b), the mixture MA is contacted with a glutathione S-transferase in an appropriate medium.

Advantageously, the weight ratio glutathione S-transferase cell pellets/mixture MA in the appropriate medium is comprised between 1 g/kg and 10 g/kg.

In particular the appropriate medium is an aqueous medium comprising glutathione. More particularly, the weight ratio glutathione/glutathione S-transferase cell pellets in the appropriate medium is comprised between 0.06 g/kg and 3 g/kg.

In particular, the appropriate medium comprises glycine. More particularly, the weight ratio of glycine/glutathione S-transferase cell pellets in the appropriate medium is comprised between 1 g/kg and 50 g/kg.

Preferably, the appropriate medium has a pH comprises between 6 and 11, more preferably between 8 and 10.

Preferably, the mixture MA is contacted with glutathione S-transferase for at least 50 hours, more preferably at least 60 hours.

In particular, the mixture MA is contacted with glutathione S-transferase at a temperature comprised between 20° C. and 45° C.

Thus, in step b) mixture MA is contacted with a glutathione S-transferase preferably in an aqueous medium comprising glutathione, optionally glycine, preferably at a pH comprised between 6 and 11, preferably at a temperature comprised between 20° C. and 45° C., and preferably for at least 50 hours.

In particular, step b) comprises the following steps:
b1) adding in a reactor, preferably successively, mixture MA, water, glutathione, optionally glycine, and the glutathione S-transferase,
b2) stirring the mixture obtained after step b1) for at least 50 hours at a temperature comprises between 20° C. and 45° C.

The mixture obtained after step b1) corresponds to the mixture MA contacted to the glutathione S-Transferase in the appropriate medium mentioned above.

The mixture obtained after step b) is called mixture MB.

Resulting mixture MB has a pH comprised between 5 and 7.

1.3 Step c)

Step c) comprises an enzymatic treatment which comprises a step of contacting mixture MA or mixture MB with a para-hydroxybenzoate hydroxylase to obtain a mixture MC.

In the sense of the invention, a Para-hydroxybenzoate hydroxylase, classified as EC 1.14.13.2, is a flavoprotein involved in degradation of aromatic compounds. The cofactor NADP is involved in the reaction as an acceptor of hydrogen, and is transformed into NADPH during the reaction. In step c), this enzyme catalyzes the addition of hydroxyl groups over the aromatic moieties, highly present in resins structure, as presented schematically below:

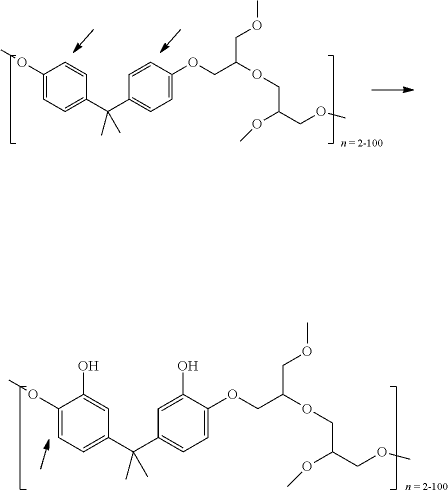

Therefore, this reaction allows an oxidation of the resin. The resin is then more hydrophilic, and its solubility in aqueous medium is enhanced. Moreover, these hydroxylated moieties are more susceptible to the cleavage performed in step d).

In a preferred embodiment, the para-hydroxybenzoate hydroxylase (PHBH) used in step c) is issued from Pseudomonas aeruginosa. In another embodiment, the para-hydroxybenzoate hydroxylase (PHBH) used in step c) is issued from Corynebacterium glutamicum.

The PHBH enzyme used in step c) may be a wild-type enzyme or a mutated one.

For this step c), several mutants of PHBH were evaluated (see example C). Although all tested enzymes were able to catalyze the hydroxylation of the resin, one of them was identified as being particularly efficient: the PHBH from Pseudomonas aeruginosa, modified with two point mutations: L199V and Y385F. This mutated enzyme has been previously reported in the application JP 2009213392 (SEQ ID NO. 10).

In a preferred embodiment of the invention, PHBH used in step c) is a mutated enzyme, even more preferentially it is a mutated enzyme presenting at least two point mutations L199V or L200V and Y385F, more preferentially is one of the following:
the mutated PHBH enzyme issued from Pseudomonas aeruginosa, modified with two point mutations: L199V and Y385F (designated in the examples as “M010”), or
the mutated PHBH enzyme issued from Corynebacterium glutamicum, modified with both point mutations: L200V and Y385F (designated in the examples as “YM321”), or
the mutated PHBH enzyme issued from Corynebacterium glutamicum, modified with three point mutations: L200V, Y385F and D39Y (designated in the examples as “YM322”).

In particular, the PHBH can be immobilized on a suitable solid support.

In particular, in step c), the previous mixture (MA or MB) is contacted with a PHBH in an appropriate medium, in particular in an aqueous medium comprising an appropriate amount of the cofactor nicotinamide adenine dinucleotide phosphate (NADP).

Advantageously, the aqueous medium comprises compounds allowing the regeneration of the cofactor NADP. Means for the regeneration of NADP are well known by the man skilled in the art. In particular, the appropriate medium for step c) may comprise glucose and an enzyme with glucose dehydrogenase activity, which reduces NADP⁺ to NADPH while oxidizing glucose-6-phosphate (classified in EC 1.1.1.49). The man skilled in the art knows well these enzymes and will be able to choose one glucose dehydrogenase such as ET004.

Preferentially, the aqueous medium comprises flavine adenine dinucleotide (FAD).

More preferentially, the aqueous system comprises glucose dehydrogenase such as ET004 and FAD.

Advantageously, the PHBH is added in the form of a broth of lysed bacteria expressing PHBH.

The bacteria are as described above. The broth of lysed bacteria is obtained by cultivation of bacteria as described above followed by centrifugation to obtain cell pellets, then cell pellets are mixed with phosphate buffer (0.1M pH=7.0) in a proportion of 1 g (of pellets)/10 ml (of buffer) and bacteria are lysed by sonication.

The broth obtained from the bacteria lysis contains the PHBH. At least 1 vol. % of this solution can be used, preferably from 1 vol. % to 20 vol. % can be used.

Step c) can be performed after step b) or concomitantly with step b).

If step c) is performed after step b), then step c) comprises an enzymatic treatment which comprises a step of contacting mixture MB with a para-hydroxybenzoate hydroxylase to obtain a mixture MC.

If step c) is performed concomitantly with step b), then step c) comprises an enzymatic treatment which comprises a step of contacting mixture MA with a para-hydroxybenzoate hydroxylase to obtain a mixture MC.

In a first embodiment, in the method according to the invention, step b) is followed by a successive step c) which consists in treating enzymatically mixture MB obtained after step b), the treatment comprising a step of contacting mixture MB with a para-hydroxybenzoate hydroxylase to obtain a mixture MC.

In this embodiment, enzymatic treatment in the method according to the invention comprises performing step b) followed by step c).

Advantageously, the weight ratio PHBH cell pellets/mixture B in the appropriate medium is comprised between 1 g/kg and 10 g/kg.

In particular, the appropriate medium is an aqueous medium comprising NADP. More particularly, the weight ratio NADP/PHBH cell pellets in the appropriate medium is comprised between 0.1 g/kg and 10 g/kg.

In particular, the appropriate medium comprises glucose. More particularly, the weight ratio of glucose/PHBH cell pellets in the appropriate medium is comprised between 50 g/kg and 300 g/kg.

Advantageously, the appropriate medium comprises FAD. More particularly, the weight ratio of FAD/PHBH cell pellets in the appropriate medium is comprised between 0.0002 g/kg and 0.002 g/kg.

In particular, the appropriate medium comprises a glucose dehydrogenase such as ET004. More particularly, the weight ratio of ET004 cell pellets/PHBH cell pellets in the appropriate medium is comprised between 0.3 g/kg and 10 g/kg.

Preferably, the appropriate medium has a pH comprised between 6 and 11, more preferably between 8 and 9.

Preferably, mixture MB is contacted with PHBH for at least 50 hours under atmospheric air pressure.

In particular, mixture MB is contacted with PHBH at a temperature comprised between 20° C. and 45° C., more preferably between 30° C. and 40° C.

Thus, in step c) mixture MB is contacted with a PHBH preferably in an aqueous medium comprising NADP, optionally glucose, optionally FAD, optionally ET004, preferably at a pH comprised between 8 and 9, preferably at a temperature comprised between 20° C. and 45° C., and preferably for at least 50 hours.

In particular, step c) comprises the following steps:
c1) adding in a reactor, preferably successively, mixture MB, glucose, NADP, FAD, ET004 and PHBH,
c2) stirring the mixture obtained after step c1) for at least 50 hours at a temperature comprised between 20° C. and 45° C.

The mixture obtained after step c1) corresponds to the mixture MB contacted to the PHBH in the appropriate medium mentioned above.

In a second embodiment, in the method according to the invention, step b) is concomitant to step c). Thus, step a) is followed by an enzymatic treatment of mixture MA obtained after step a), the treatment comprising a step of contacting mixture MA with a glutathione S-transferase and a para-hydroxybenzoate hydroxylase to obtain a mixture MC.

In this embodiment, enzymatic treatment in the method according to the invention comprises performing step b) and step c) concomitantly.

In particular, when step b) and step c) are performed concomitantly, mixture MA is contacted with a GST and a PHBH concomitantly in an appropriate medium.

Advantageously, the weight ratio GST cell pellets/mixture A in the appropriate medium is comprised between 1 g/kg and 10 g/kg.

Advantageously, the weight ratio PHBH cell pellets/mixture A in the appropriate medium is comprised between 1 g/kg and 10 g/kg.

In particular, the appropriate medium is an aqueous medium comprising NADP and glutathione. More particularly, the weight ratio NADP/PHBH cell pellets in the appropriate medium is comprised between 0.1 g/kg and 10 g/kg. More particularly, the weight ratio glutathione/GST cell pellets in the appropriate medium is comprised between 0.06 g/kg and 3 g/kg.

In particular, the appropriate medium comprises glycine. More particularly, the weight ratio of glycine/glutathione S-transferase cell pellets in the appropriate medium is comprised between 1 g/kg and 50 g/kg.

In particular, the appropriate medium comprises glucose. More particularly, the weight ratio of glucose/PHBH cell pellets in the appropriate medium is comprised between 50 g/kg and 300 g/kg.

Advantageously, the appropriate medium comprises FAD. More particularly, the weight ratio of FAD/PHBH cell pellets in the appropriate medium is comprised between 0.0002 g/kg and 0.02 g/kg.

In particular, the appropriate medium comprises ET004. More particularly, the weight ratio of ET004 cell pellets/PHBH cell pellets in the appropriate medium is comprised between 0.3 g/kg and 10 g/kg.

Preferably, the appropriate medium has a pH comprised between 6 and 11, more preferably between 8 and 9.

Preferably, mixture MA is contacted with GST and PHBH for at least 50 hours.

In particular, mixture MA is contacted with GST and PHBH at a temperature comprised between 20° C. and 45° C.

Thus, when step b) and step c) are performed concomitantly, mixture MA is contacted with a GST and a PHBH preferably in an aqueous medium comprising glutathione, NADP, optionally glucose, optionally FAD, optionally ET004, preferably at a pH comprised between 6 and 11, preferably at a temperature comprised between 20° C. and 45° C., and preferably for at least 50 hours.

In particular, step b) and step c) comprise the following steps:
bc1) adding in a reactor, preferably successively, mixture MA, water, glucose, glutathione, glycine, NADP, FAD, ET004, PHBH and GST,
bc2) stirring the mixture obtained after step bc1) for at least 96 hours at a temperature comprised between 20° C. and 45° C.

The mixture obtained after step bc1) corresponds to the mixture MA contacted to the GST and the PHBH concomitantly in the appropriate medium mentioned above.

In both embodiments, the mixture obtained after step c) is called mixture MC.

Resulting mixture MC has a pH comprised between 5 and 7.

1.4 Step d)

step d) comprises treating chemically mixture MB or mixture MC, the chemical treatment comprising a step of contacting mixture MB or mixture MC with an aqueous solution comprising a strong Bronsted base to obtain a mixture MD.

Indeed, step d) is performed after enzymatic treatment.

Enzymatic treatment can comprise performing step b) to obtain mixture MB or performing step b) and step c) successively or concomitantly to obtain mixture MC.

Thus, step d) can be performed after step b). Then step d) comprises treating chemically mixture MB, the chemical treatment comprising a step of contacting mixture MB with an aqueous solution comprising a strong Bronsted base to obtain a mixture MD. In this embodiment, step c) is not performed.

Alternatively, step d) can be performed after step c). Then step d) comprises treating chemically mixture MC, the chemical treatment comprising a step of contacting mixture MC with an aqueous solution comprising a strong Bronsted base to obtain a mixture MD. In this embodiment, step b) and step c) are performed successively or concomitantly.

In particular, in both embodiments, the strong Bronsted base is selected from hydroxides of alkali metals and alkaline earth metals such as sodium hydroxide, lithium hydroxide or potassium hydroxide. Preferably, the strong Bronsted base is sodium hydroxide.

Preferably, the strong Bronsted base is in a concentration comprised between 0.1 mol/L and 10 mol/L, more preferably between 1 mol/L and 10 mol/L.

Advantageously, the weight ratio (aqueous solution of a strong Bronsted base)/mixture MB or the weight ratio (aqueous solution comprising a strong Bronsted base)/mixture MC is comprised between 20 g/g and 1 g/g.

Advantageously, in each embodiment, in step d) mixture MB or mixture MC is in contact with the aqueous solution comprising a strong Bronsted base for 48 hours to 2 weeks at a temperature comprised between 20° C. and 100° C., more advantageously at a temperature between 60° C. and 100° C.

2. Rounds

The method according to the invention can be reproduced several times, preferably at least one time, more preferably at least two times. In these reproductions, step a) is optional.

The method of the invention can be reproduced between one time and 30 times, preferably between one time and 20 times, more preferably one time and ten times.

A first round of the method according to the invention corresponds to:

a) adding in a solvent an epoxy resin R based on:
at least one aromatic compound R1 bearing at least two epoxide groups per molecule and comprising at least one aromatic ring bearing at least one glycidyloxy group, and
at least one curing agent R2,

b) treating enzymatically the mixture MA obtained after step a), the treatment comprising a step of contacting the mixture MA with a glutathione S-transferase to obtain a mixture MB,

c) optionally treating enzymatically mixture MA concomitantly to step b) or treating enzymatically mixture MB obtained after step b), the treatment comprising a step of contacting mixture MA or mixture MB with a para-hydroxybenzoate hydroxylase to obtain a mixture MC,

d) optionally treating chemically mixture MB or mixture MC, the chemical treatment comprising a step of contacting mixture MB or mixture MC with an aqueous solution comprising a strong Bronsted base to obtain a mixture MD.

The mixture M1 obtained after performing a first round of the method according to the invention can be submitted again to the method according to the invention and thus replace the epoxy resin R in step a) or mixture MA in step b). A second round of the method according to the invention is then performed.

Thus, the second round corresponds to:

a) optionally adding in a solvent, the mixture M1 obtained after a first round of the method according to the invention,

b) treating enzymatically the mixture MA′ obtained after step a) or the mixture M1 obtained after a first round, the treatment comprising a step of contacting the mixture MA′ or M1 with a glutathione S-transferase to obtain a mixture MB′,

c) optionally treating enzymatically mixture MA′ or M1 concomitantly to step b) or treating enzymatically mixture MB′ obtained after step b), the treatment comprising a step of contacting mixture MA′ or M1 or mixture MB′ with a para-hydroxybenzoate hydroxylase to obtain a mixture MC′,

d) optionally treating chemically mixture MB′ or mixture MC′, the chemical treatment comprising a step of contacting mixture MB′ or mixture MC′ with an aqueous solution comprising a strong Bronsted base to obtain a mixture MD′.

The mixture M1 obtained after performing a first round of the method according to the invention can be mixture MB, mixture MC or mixture MD as defined above. Preferably, it corresponds to mixture MD.

Advantageously, mixture MD obtained after step d) is then further submitted to steps b) to d) as defined above.

Alternatively, mixture MD obtained after step d) is further submitted to steps a) to d) as defined above.

In particular, more than two rounds can be performed, more particularly more than three rounds can be performed. Preferably, from 1 to 30 rounds can be performed, more preferably from 2 to 20 rounds, even more preferably from 2 to 10 rounds.

Advantageously, one to ten rounds of the method according to the invention can be performed.

3. Epoxy Resin

The method according to the invention aims at degrading epoxy resins.

The epoxy resin used in the method of the invention is based on:
at least one aromatic compound R1 bearing at least two epoxide groups per molecule and comprising at least one aromatic ring bearing at least one glycidyloxy group, and
at least one curing agent R2,
optionally an additional compound R3.

The expression “epoxy resin based on” should, of course, be understood as meaning an epoxy resin comprising the mixture and/or the reaction product of the various base constituents used for this composition, it being possible for some of them to be intended to react or capable of reacting with one another or with their immediate chemical surroundings, at least partly, during the various phases of manufacture of the epoxy resin, or of the composites or finished articles comprising such composites, in particular during a curing step.

In other words, the epoxy resin R is manufactured from at least one aromatic compound R1 as described below and at least one curing agent R2.

Advantageously, the epoxy resin R is based on one aromatic compound R1 and one curing agent R2.

As used herein in reference to an organic compound, the term “aromatic” means that the organic compound that comprises one or more one aryl moieties, which may each optionally be interrupted by one or more heteroatoms, typically selected from oxygen, nitrogen, and sulfur heteroatoms, and one or more of the carbon atoms of one or more one aryl moieties may optionally be substituted with one or more organic groups, typically selected from alkyl, alkoxyl, hydroxyalkyl, cycloalkyl, alkoxyalkyl, haloalkyl, aryl, alkaryl, aralkyl.

As used herein, the term “aryl” means cyclic, coplanar 5- to 14-membered organic group having a delocalized, conjugated π system, with a number of π electrons that is equal to 4n+2, where n is 0 or a positive integer, including compounds where each of the ring members is a carbon atom, such as benzene, compounds where one or more of the ring members is a heteroatom, typically selected from oxygen, nitrogen and sulfur atoms, such as furan, pyridine, imidazole, and thiophene, and fused ring systems, such as naphthalene, anthracene, and fluorene, wherein one or more of the ring carbons may be substituted with one or more organic groups, typically selected from alkyl, alkoxyl, hydoxyalkyl, cycloalkyl, alkoxyalkyl, haloalkyl, aryl, alkaryl, halo groups, such as, for example, phenyl, methylphenyl, trimethylphenyl, nonylphenyl, chlorophenyl, or trichloromethylphenyl.

As used herein, “epoxide group” means a vicinal epoxy group, i.e., a 1,2-epoxy group.

3.1. Aromatic Compound R1

The first essential compound of the epoxy resin R is an aromatic compound R1 bearing at least two epoxide groups per molecule and comprising at least one aromatic ring bearing at least one glycidyloxy group.

The aromatic compound R1 according to the invention bears at least two epoxide groups per molecule, and one of the at least two epoxide groups might be the epoxide group from the glycidyloxy group bore by the aromatic ring bearing at least one glycidyloxy group.

In particular suitable aromatic compounds R1 include polyglycidyl ethers of phenols and of polyphenols, such as diglycidyl resorcinol, 1,2,2-tetrakis(glycidyloxyphenyl) ethane, or 1,1,1-tris(glycidyloxyphenyl)methane, diglycidyl ether of bisphenol, such as diglycidyl ether of bisphenol A (bis(4-hydroxyphenyl)-2,2-propane), diglycidyl ether of bisphenol F (bis(4-hydroxyphenyl)methane), diglycidyl ether of bisphenol C (bis(4-hydroxyphenyl)-2,2-dichloroethylene), and diglycidyl ether of bisphenol S (4,4′-sulfonyldiphenol), including oligomers thereof, polyglycidyl ethers of aromatic alcohols, epoxidized novolac compounds, epoxidized cresol novolac compounds, polyglycidyl ether of aminophenols such as triglycidyl aminophenols (TGAP), triglycidyl aminocresol.

Preferably, suitable aromatic compounds R1 include known, commercially available compounds, such as triglycidyl ethers of p-aminophenol (such as MY 0510 from Hunstman); triglycidyl ethers of m-aminophenol (such as MY 0610 from Hunstman); diglycidyl ethers of bisphenol A based materials such as 2,2-bis(4,4′-dihydroxy phenyl) propane (such as DER 661 from Dow, or EPON 828 from Momentive); glycidyl ethers of phenol Novolac resins (such as. DEN 431 or DEN 438 from Dow); diglycidyl derivative of dihydroxy diphenyl methane (such as PY 306 from Huntsman).

Advantageously, the aromatic compound R1 corresponds to a diglycidyl ether of bisphenol, such as diglycidyl ether of bisphenol A (bis(4-hydroxyphenyl)-2,2-propane), diglycidyl ether of bisphenol F (bis(4-hydroxyphenyl)methane), diglycidyl ether of bisphenol C (bis(4-hydroxyphenyl)-2,2-dichloroethylene), and diglycidyl ether of bisphenol S (4,4′-sulfonyldiphenol).

In particular the aromatic compound R1 corresponds to a bisphenol A diglycidyl ether.

3.2 Curing Agent R2

The second essential compound of the epoxy resin R is the curing agent R2.

Curing agents of epoxy resins are well-known to one skilled in the art.

It can be an amine, such as a primary amine, a secondary amine, or a tertiary amine, a ketimine, a polyamide resin, an imidazole derivative, a polymercaptan, an anhydride, a boron-trifluoride-amine complex, a dicyandiamide, an organic acid hydrazide, a photocuring agent or an ultraviolet-curing agent.

In particular, amine as curing agent is a polyamine. It can be an aliphatic polyamine or an aromatic amine.

Suitable amine as curing agent include diethylenetriamine (DTA), triethylenetetramine (TTA), tetraethylenepentamine (TEPA), dipropenediamine (DPDA), diethylaminopropylamine (DEAPA), amine 248, N-aminoethylpiperazine (N-AEP), Lamiron C-260, Araldit HY-964, menthan diamine (MDA), isophoronediamine (IPDA), S cure 211, Wandamin HM, 1.3 BAC, m-xylenediamine (m-XDA), Sho-amine X, Amine black, Sho-amine black, Sho-amine N, Sho-amine 1001, Sho-amine 1010, metaphenylene diamine (MPDA), diaminodiphenylmethane (DDM), diaminodiphenylsulfone (DDS), 4,4′-methylenebis(2,6-diethylaniline.

Suitable imidazole derivative as curing agent R2 include 2-methylimidazole, 2-phenylimidazole, 3-benzyl-2-methylimidazole, 5-methyl-2-phenylimidazole, 2-ethyl methylimidazole, 5-ethyl-2-methylimidazole or 1-cyanoethyl-2-undecylimidazolium trimellitate.

Suitable anhydride as curing agent R2 include phthalic anhydride, trimellitic anhydride, pyromellitic anhydride, benzophenone tricarboxylic anhydride, ethylene glycol bistrimellitate, glycerol tristrimellitate, maleic anhydride, tetrahydrophthalic anhydride, enomethylene tetrahydrophthalic anhydride, methylendomethyldene tetrahydrophthalic anhydride, dodecenyl succinic anhydride, hexahydrophthalic anhydride, hexahydro-4-methylphthalic anhydride, succinic anhydride, methylcyclohexene dicarboxylic anhydride, alkylstryrene-maleic anhydride copolymer, chlorendic anhydride, polyazelaic polyanhydride.

Preferably, the curing agent R2 is chosen from a primary amine and an imidazole derivative.

3.3 Additional Compound R3

The epoxy resin R can also be based on at least one epoxy compound that has at least two epoxide groups per molecule. Suitable epoxy compounds include aromatic epoxy compounds, epoxy compounds, alicyclic epoxy compounds, and epoxy compounds.

Suitable aromatic epoxy compounds include aromatic compounds having two or more epoxide groups per molecule, including known compounds such as, for example: polyglycidyl ethers of phenols and of polyphenols, such as diglycidyl resorcinol, 1,2,2-tetrakis(glycidyloxyphenyl) ethane, or 1,1,1-tris(glycidyloxyphenyl)methane, the diglycidyl ethers of bisphenol A (bis(4-hydroxyphenyl)-2,2-propane), bisphenol F (bis(4-hydroxyphenyl)methane), bisphenol C (bis(4-hydroxyphenyl)-2,2-dichloroethylene), and bisphenol S (4,4′-sulfonyldiphenol), including oligomers thereof, fluorene ring-bearing epoxy compounds, naphthalene ring-bearing epoxy compounds, dicyclopentadiene-modified phenolic epoxy compounds, epoxidized novolac compounds, and epoxidized cresol novolac compounds, polyglycidyl adducts of amines, such as N,N-diglycidyl aniline, N,N,N′,N′-tetraglycidyl diaminodiphenylmethane (TGDDM), triglycidyl aminophenols (TGAP), triglycidyl aminocresol, or tetraglycidyl xylenediamine, or amino alcohols, such as triglycidyl aminophenol, polyglycidyl adducts of polycarboxylic acids, such as diglycidyl phthalate, polyglycidyl cyanurates, such as triglycidyl cyanurate, copolymers of glycidyl(meth)acrylates with copolymerizable vinyl compounds, such as styrene glycidyl methacrylate.

Suitable epoxy compounds having two or more epoxide groups per molecule include known, commercially available compounds, such as N,N,N′,N′-tetraglycidyl diamino diphenylmethane (such as MY 9663, MY 720, and MY 721 from Huntsman), N,N,N′,N′-tetraglycidyl-bis(4-aminophenyl)-1,4-diiso-propylbenzene (such as EPON 1071 from Momentive); N,N,N′,N′-tetraclycidyl-bis(4-amino-3,5-dimethylphenyl)-1,4-diisopropylbenzene, (such as EPON 1072 from Momentive); triglycidyl ethers of p-aminophenol (such as MY 0510 from Hunstman); triglycidyl ethers of m-aminophenol (such as MY 0610 from Hunstman); diglycidyl ethers of bisphenol A based materials such as 2,2-bis(4,4′-dihydroxy phenyl) propane (such as DER 661 from Dow, or EPON 828 from Momentive, and Novolac resins preferably of viscosity 8-20 Pas at 25° C.; glycidyl ethers of phenol Novolac resins (such as. DEN 431 or DEN 438 from Dow); di-cyclopentadiene-based phenolic novolac (such as Tactix® 556 from Huntsman); diglycidyl 1,2-phthalate; diglycidyl derivative of dihydroxy diphenyl methane (such as PY 306 from Huntsman).

Suitable alicyclic epoxy compounds having two or more epoxide groups per molecule, including known compounds such as, for example, bis(2,3-epoxy-cyclopentyl)ether, copolymers of bis(2,3-epoxy-cyclopentyl)ether with ethylene glycols, dicyclopentadiene diepoxide, 4-vinyl cyclohexene dioxide, 3,4-epoxycyclohexylmethyl, 3,4-epoxycyclohexane carboxylate, 1,2,8,9-diepoxy limonene (limonene dioxide), 3,4-epoxy-6-methyl-cyclohexylmethyl, 3,4-epoxy-6-methylcyclohexane carboxylate, bis(3,4-epoxy-6-methylcyclohexylmethyl)adipate, 2-(7-oxabicyclo[4.1.0]hept-3-yl)spiro[1,3-dioxane-5,3′-[7]oxabicyclo[4.1.0]heptane], diepoxides of allyl cyclopentenyl ether, 1,4-cyclohexadiene diepoxide, 1,4-cyclohexanemethanol diglydical ether, bis(3,4-epoxycyclohexylmethyl) adipate, 3,4-epoxy-6-methylcyclohexane carboxylate, diglycidyl 1,2-cyclohexane carboxylate, 3,4-epoxycyclohexylmethyl methacrylate, 3-(oxiran-2-yl)-7-oxabicyclo[4.1.0]heptane, bis(2,3-epoxypropyl) cyclohex-4-ene-1,2-dicarboxylate, 4,5-epoxytetrahydrophthalic acid diglycidyl ester, poly[oxy(oxiranyl-1,2-cyclohexanediyl)] α-hydro-ω-hydroxy-ether, bi-7-oxabicyclo[4.1.0]heptane.

Suitable aliphatic epoxy compounds having two or more epoxide groups per molecule, including known compounds such as, for example: butanediol diglycidyl ether, epoxidized polybutadiene, dipentene dioxide, trimethylolpropane triglycidyl ether, bis[2-(2-butoxyethyoxy)ethyl)ethyl] adipate, hexanediol diglycidyl ether, and hydrogenated bisphenol A epoxy resin.

Suitable alicyclic epoxy compounds and aliphatic epoxy compounds include known, commercially available compounds, such as, for example: 3′,4′-epoxycyclohexanemethyl-3,4-epoxycyclohexylcarboxylate (CELLOXIDE™ 2021P resin (Daicel Corporation) and ARADITE CY 179 (Huntsman Advanced Materials)), bi-7-oxabicyclo[4.1.0]heptane (CELLOXIDE™ 8010 (Daicel Corporation)) 3:1 mixture of poly[oxy(oxiranyl-1,2-cyclohexanediyl)], α-hydro-ω-hydroxy-ether with 2-ethyl-2-(hydroxymethyl)-1,3-propanediol (EHPE 3150 (Daicel)).

The epoxy resin may optionally further be based on one or more monoepoxide compounds having one epoxide group per molecule, selected from aromatic monoepoxy compounds, monoalicyclic epoxy compounds, and aliphatic monoepoxy compounds. Suitable monoepoxide compounds, including known compounds such as, for example: saturated alicylic monoepoxides, such as 3,3′-bis(chloromethyl)oxacyclobutane, isobutylene oxide, styrene oxide, olefinic monoepoxides, such as cyclododecadiene monoepoxide, 3,4-epoxy butene.

4. Uses

The present invention also deals with the use of a glutathione S-transferase, in particular a glutathione S-transferase issued from a N. aromaticivorans strain, for degrading an epoxy resin R based on:
at least one aromatic compound R1 bearing at least two epoxide groups per molecule and comprising at least one aromatic ring bearing at least one glycidyloxy group, and
at least one curing agent R2.

The glutathione S-transferase is as described above in paragraph “step b)”.

The epoxy resin R corresponds to an epoxy resin as used in the method of the invention and as described above in the paragraph “epoxy resin”.

Additionally, the present invention relates to the use of a para-hydroxybenzoate hydroxylase, preferably a para-hydroxybenzoate hydroxylase issued from Pseudomonas aeruginosa, more preferably a mutated para-hydroxybenzoate hydroxylase presenting the peptide sequence as shown in SEQ ID NO. 1, for degrading an epoxy resin R based on:
at least one aromatic compound R1 bearing at least two epoxide groups per molecule and comprising at least one aromatic ring bearing at least one glycidyloxy group, and
at least one curing agent R2.

The para-hydroxybenzoate hydroxylase is as described above in paragraph “step c)”.

The epoxy resin R corresponds to an epoxy resin as used in the method of the invention and as described above in the paragraph “epoxy resin”.

5. Method for Recycling a Composite Material

The present invention deals with method for recycling a composite material comprising the following steps:

a′) providing a composite material CM comprising an epoxy resin R based on:
at least one aromatic compound R1 bearing at least two epoxide groups per molecule and comprising at least one aromatic ring bearing at least one glycidyloxy group, and
at least one curing agent R2,

b′) treating the composite material CM obtained after step a′), the treatment comprising a step of degrading the epoxy resin R with the method according to the invention, in which case the epoxy resin R in step a) is the composite material CM,

c′) obtaining after step b′) the composite material with a reduced content in epoxy resin.

Thus, in step b′), the step of degrading the epoxy resin R corresponds to:
a) adding in a solvent a composite material comprising an epoxy resin R according to the invention to obtain mixture MA,
b) treating enzymatically the mixture MA obtained after step a), the treatment comprising a step of contacting the mixture MA with a glutathione S-transferase to obtain a mixture MB,
c) optionally treating enzymatically mixture MA concomitantly to step b) or treating enzymatically mixture MB obtained after step b), the treatment comprising a step of contacting mixture MA or mixture MB with a para-hydroxybenzoate hydroxylase to obtain a mixture MC,
d) optionally treating chemically mixture MB or mixture MC, the chemical treatment comprising a step of contacting mixture MB or mixture MC with an aqueous solution comprising a strong Bronsted base to obtain a mixture MD.

A composite material is a material made from two or more constituent materials with significantly different physical or chemical properties that, when combined, produce a material with characteristics different from the individual components. Thus, typically, a composite material comprises a matrix and reinforcement.

In the present invention, the composite material comprises reinforcement and a matrix which comprises at least one epoxy resin R as described above.

The reinforcement is preferably reinforcing fibers. It can be mineral, organic or plant fibers, notably glass fibers or carbon fibers. More preferably, the reinforcement is carbon fibers.

The matrix comprises at least one epoxy resin R as described above. Thus, the matrix can comprise one or more epoxy resin as described above.

The matrix can also comprise other thermosetting resins, such as unsaturated polyesters, polyvinyl esters, phenolic resin and polyurethanes.

The matrix can also comprise another epoxy resin based on at least one additional compound R3 as described above and at least one curing agent R2 as described above.

The matrix can also comprise thermoplastic resins such as polyetherimide (PEI), polyether sulfone (PES), polysulfone, polyamideimide (PAI), polyamide (PA), polyimide, polyphenylene sulfide (PPS), polyetheretherketone (PEEK), polyolefin or combinations thereof.

Advantageously, the matrix comprises polyamide particles, polyimide particles or combination thereof.

In particular, the matrix comprises polyetherimide, polyether sulfone or a combination thereof. These thermoplastic resins can be used as toughening agent. The matrix is then a thermoplastic toughened epoxy resin.

The matrix can also include accelerators enhancing or promoting the curing of the epoxy resin.

The matrix can comprise performance modifying agents such as core shell rubbers, flame retardants, wetting agents, pigments, dyes, UV absorbers, fillers, conducting particles and viscosity modifiers.

Preferably, the epoxy resin is the main constituent of the matrix. More preferably, it represents at least 50 wt. % of the matrix.

As described in the paragraph “rounds” above, the mixture obtained after step b′), after performing a first round of the method for degrading an epoxy resin according to the invention can be submitted again to the method for degrading an epoxy resin according to the invention and thus replace the composite material CM in step b′). A second round of the method according to the invention is then performed. Several rounds can be performed int his manner.

Then the method for recycling a composite material comprises the following steps:

a′) providing a composite material CM comprising an epoxy resin R based on:
at least one aromatic compound R1 bearing at least two epoxide groups per molecule and comprising at least one aromatic ring bearing at least one glycidyloxy group, and
at least one curing agent R2,

b1′) treating the composite material CM obtained after step a′), the treatment comprising a step of degrading the epoxy resin R with the method according to the invention, in which case the epoxy resin R in step a) is the composite material CM,

b2′) repeating step b1′) by treating the composite material with a reduced content in epoxy resin obtained after step b1′), the treatment comprising a step of degrading the epoxy resin R with the method according to the invention, in which case the epoxy resin R in step a) is the composite material with a reduced content in epoxy resin obtained after step b1′),

c′) obtaining after step b′) the composite material with a reduced content in epoxy resin.

Advantageously, the composite material CM comprises carbon fibers. Thus, in step c′), the composite material with a reduced content in epoxy resin is obtained and carbon fibers can be isolated.

The method for recycling a composite material according to the invention allows then to recycle carbon fibers from the composite material.

### EXAMPLES

Epoxy resins tested in the examples are as follows:

Resin R1 is obtained by polymerization of Bisphenol A diglycidyl ether (also noted BADGE or DGEBA) in the presence of 4,4′-Methylenebis(2,6-diethylaniline) and has the following general structure:

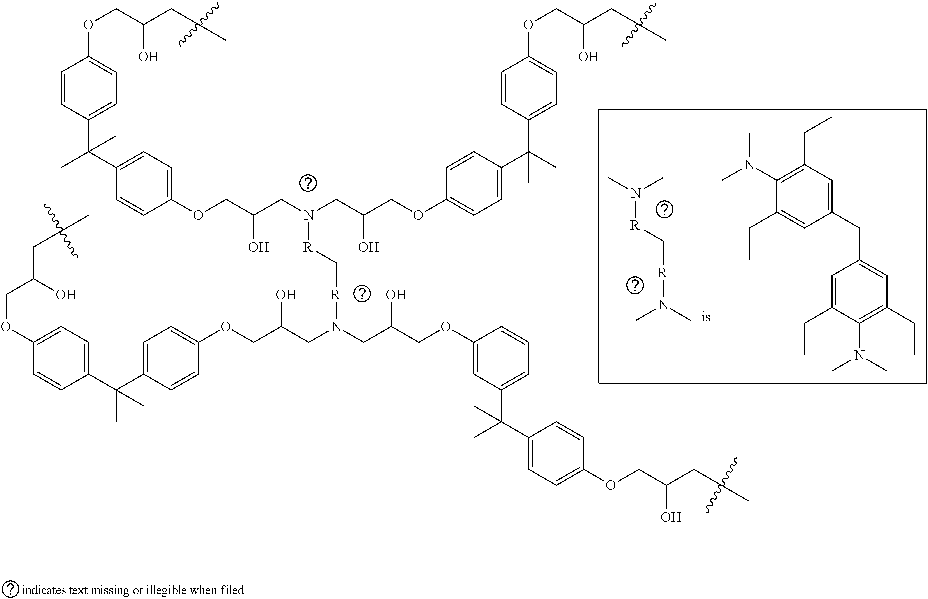

Resin R2 is obtained by polymerization of Bisphenol A diglycidyl ether (also noted BADGE or DGEBA) in the presence of an imidazole derivative (5-ethyl-2-methylimidazole) and has the following general structure:

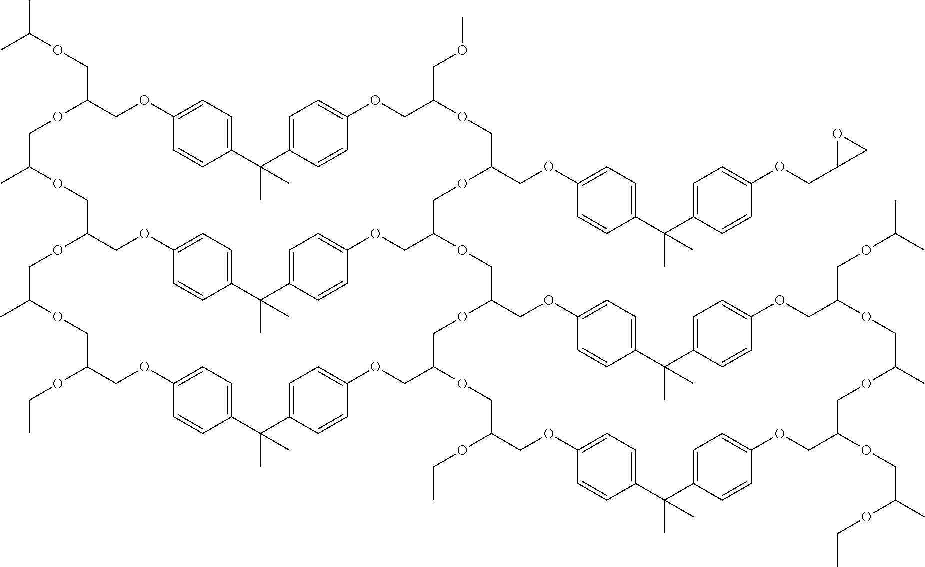 [SMILES: CCOC(C)COC1COc2ccc(cc2)C(C)(C)c2ccc(cc2)OCC(OCC)COC2COc3ccc(cc3)C(C)(C)c3ccc(cc3)OCC(OCC)COC(C)COC3COc4ccc(cc4)C(C)(C)c4ccc(cc4)OCC(COC(COc4ccc(C(C)(C)c5ccc(OCC6CO6)cc5)cc4)COC(COC)COc4ccc(cc4)C(C)(C)c4ccc(cc4)OCC(COC(C)C)OCC(C)OC3)OCC(COc3ccc(cc3)C(C)(C)c3ccc(cc3)OCC(COC(C)C)OCC(C)OC1)OC2]

In the examples, GST is added as a broth of lysed GST-expressing bacteria. It is produced following the process:

1. growing GST-expressing strain and centrifuging to obtain the cell pellets;

2. mixing cell pellets with phosphate buffer (0.1M pH=7.0) in a proportion of 1 g (of pellets)/10 ml (of buffer);

3. Lysing cells by sonication in the phosphate buffer to obtain a broth containing the GST.

In the examples, PHBH is added as a broth of lysed PHBH-expressing bacteria. It is produced following the process:

1. growing PHBH-expressing strain and centrifuging to obtain the cell pellets;

2. mixing cell pellets with phosphate buffer (0.1M pH=7.0) in a proportion of 1 g (of pellets)/10 ml (of buffer);

3. Lysing cells by sonication in the phosphate buffer to obtain a broth containing the PHBH.

In the examples, ET004 is added as a broth of lysed ET004-expressing bacteria. It is produced following the process:

1. growing ET004-expressing strain and centrifuging to obtain the cell pellets;

2. mixing cell pellets with phosphate buffer (0.1M pH=7.0) in a proportion of 1 g (of pellets)/10 ml (of buffer);

3. Lysing cells by sonication in the phosphate buffer to obtain a broth containing the ET004.

A—Step a) Adding an Epoxy Resin to a Solvent

This step aims at swelling and potentially decrosslinking the epoxy resin in order to improve the accessibility to the key bond sites for the enzymes used in the following steps.

First, a series of solvents have been tested (water, an aqueous solution of Na₂HPO₄, an aqueous solution of H₃PO₄, an aqueous solution of ammonia, acetic acid, DMSO, DMF, hexane and phenol).

The test consists in soaking an epoxy resin R1 or R2 with a solvent for 80 days at room temperature in resin/solvent proportions of 0.1 g/1 ml. These preliminary assays were conducted to evaluate the behavior of the materials once exposed to these solvents.

The aqueous solution of Na₂HPO₄ was selected not only for its capability of promoting modification of the resin, but also because it is safe to handle, it is cheap and it can be recycled.

The mixture obtained after step a) has been studied to evaluate what is happening during this step.

Step a) has thus been performed on resin R2 as follows:

1^{st} Step: Preparing a solution of 0.21 g Resin R2+20.56 g Na₂HPO₄*12H₂O (M.W. 358.14 g/mol). Add 98.05 g H₂O, stir at 90° C. for 1 h;

2^{nd} Step: Evaporating the solution from previous step at 80° C. under vacuum until the mixture weight goes from 119 g to 55 g;

3^{rd} Step: Diluting the previous concentrated solution with 300 g of deionized H₂O at 90° C.;

4^{th} Step: Evaporating at 80° C. under vacuum, until the weight of the mixture is reduced to 44 g. Afterwards, add 456 g of water at 90° C.

5^{th} Step: Centrifuging the solution from previous step in order to obtain a yellowish supernatant and two kinds of solids: one yellowish and similar to the original resin, and the other grayish, brittle and opaque.

By HPLC and MS analysis of the resulting mixture, it was observed that first step a) led to degradation of the resin. Products resulting from phosphorylation of the resin and/or ether bond cleavage of the resin could notably be identified.

B—Step b) Treating Enzymatically by Contacting Mixture MA with a Glutathione 5-Transferase

After performing step a) on resin R1 and R2 and obtaining respectively MA1 and MA2, step b) was performed according to the method described below in order to evaluate the activity of the enzyme issued from a N. aromaticivorans strain and named “NaLigE”.

Ethyl vanillin was also tested along with MA1 and MA2 as a model.

Step a) Preparation of mixtures MA1 and MA2:

1^{st} Step: Prepare a solution of 1.98 g Resin+50.65 g deionized H₂O; +90.00 g Na₂HPO₄.12H₂O, +189 g deionized H₂O, at 60° C. until complete solubilization of the Na₂HPO₄.12H₂O salt;

2^{nd} Step: Evaporate the solution from the previous step at 80° C. under vacuum to remove water, then add 300 g of deionized H₂O, then evaporate again until complete removal of water;

3^{rd} Step: Repeat the 2^{nd} step;

4^{th} Step: Add deionized H₂O at 90° C. to the dry solid obtained after step 3;

5^{th} Step: Add 9.96 g β-Cyclodextrin to the previous solution (total weight 142 g), and mix;

6^{th} Step: Stir the mixture at 90° C. for two days;

7^{th} Step: Dilute the mixture to obtain a volume of 200 ml;

The mixture obtained after performing step 1 to 7 on resin R1 is noted MA1.

The mixture obtained after performing step 1 to 7 on resin R2 is noted MA2.

TABLE A

| | | Volume | 2.1 wt. % | 2.48 | Aqueous | Glutathione- |
|---|---|---|---|---|---|---|
| | | or | reduced | wt. % | solution | S- |
| | | mass of | glutathione | Glycine | of NaOH | Transferase |
| ID | Substrate | substrate | (ml) | (ml) | 3M | enzyme |
| 1 | Ethyl vanillin (0.4 | 0.398 ml | 0.175 | 1.452 | 0.5 ml | 3 ml |
| | wt. % in a 0.1M | | | | | |
| | phosphate buffer | | | | | |
| | system at pH 7) | | | | | |
| 2 | MA2 | 0.5 g | 0.175 | 0.725 | 1.225 ml | 3 ml |
| 3 | MA1 | 0.5 g | 0.175 | 0.725 | 1.225 ml | 3 ml |

According to table A, in a reactor of suitable volume, the components were added in the following order: 1^{st}: substrate (ethyl-vanillin or MA1 or MA2); 2^{nd}: aqueous solution of NaOH 3M; 3^{rd}: glutathione; 4^{th}: Glycine; 5^{th}: GST enzyme. Reaction is carried out at 25° C./220 rpm, during 5 days. Samples were subdued to HPLC analysis for chromatographic profile comparison (samples were collected at time 0 h, 16 h, 40 h, 66 h and 120 h).

HPLC characterization conditions:

(1) the mobile phase

Solution A: methanol;

Solution B: 1% Formic acid in ddH2O;

A:B ratio=35%:65%

(2) column: Agilent C18, 250 mm*4.6 mm

(3) Detection wavelength: 280 nm

(4) Column temperature: 25° C.

(5) Flow Rate: 1 ml/min

Results:

By HPLC chromatography, an evolution of the peaks was observed through time with both mixtures MA1 and MA2. Three major peaks at 2.7 min, 3.1 min and 3.5 min were observed at 0 hrs. The peak at 2.7 min increases with time demonstrating further degradation of the mixtures during enzymatic treatment with a GST.

It was supposed that the mechanism of action of the glutathione S-transferase was the cleavage of aryl ether bonds. It was confirmed by running the following model reaction with ethyl vanillin:

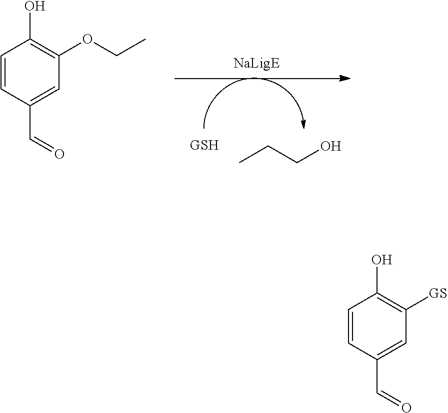

Consumption of the ethyl-vanillin substrate was indeed observed by HPLC.

The step b) of enzymatic treatment with glutathione S-transferase led to partial oligomerization of the resin.

C—Step c) treating enzymatically by contacting mixture MA with a para-hydroxybenzoate hydroxylase

After performing step a) on resins R1 and R2 and obtaining respectively mixtures MA1 and MA2, step c) was performed according to the method below in order to evaluate the activity of Mutant enzyme issued from Pseudomonas aeruginosa:
M010-2 (L199V and Y385F mutant enzyme issued from Pseudomonas aeruginosa),
M012-2 (L199G, Y385F mutant enzyme issued from Pseudomonas aeruginosa),
YM322-2 (L200V, Y385F, D39Y mutant enzyme issued from Corynebacterium glutamicum) and
M020-1 (wild-type enzyme issued from Corynebacterium glutamicum).

Step a) Preparation of mixtures MA1 and MA2:

1^{st} Step: Prepare a solution of 1.98 g Resin+50.65 g deionized H₂O; +90.00 g Na₂HPO₄.12H₂O, +189 g deionized H₂O, at 60° C. until complete solubilization of the Na₂HPO₄.12H₂O salt;

2^{nd} Step: Evaporate at 80° C. under vacuum to remove water then add 300 g of deionized H₂O, then evaporate again until complete removal of water.

3^{rd} Step: Repeat the 2^{nd} step;

4^{th} Step: Add deionized H₂O at 90° C. to the dry solid;

5^{th} Step: Add 9.96 g 13-Cyclodextrin to the previous solution (total weight 142 g), and mix;

6^{th} Step: Stir the mixture at 90° C. for two days;

7^{th} Step: Dilute the mixture to 200 ml;

The mixture obtained after performing step 1 to 7 on resin R1 is noted MA1.

The mixture obtained after performing step 1 to 7 on resin R2 is noted MA2.

TABLE B

| | | | Vol. | Glucose | ET004 | NADP | FAD (4 g/l) | PHBH |
|---|---|---|---|---|---|---|---|---|
| ID | Substrate | Enzyme | (ml) | (g) | (ml) | (50 g/l) (ml) | (μl) | (ml) |
| 1 | MA2 | None | 10 | 1.875 | 0.6 | 0.5 | 25 | 0.00 |
| 2 | MA2 | M012-2 | 10 | 1.875 | 0.6 | 0.5 | 25 | 2.00 |
| 3 | MA2 | YM322-2 | 10 | 1.875 | 0.6 | 0.5 | 25 | 3.00 |
| 4 | MA1 | M020-1 | 10 | 1.875 | 0.6 | 0.5 | 25 | 1.75 |
| 5 | MA1 | YM322-2 | 19 | 1.875 | 0.6 | 0.5 | 25 | 3.00 |
| 6 | MA1 | M010-2 | 10 | 1.875 | 0.6 | 0.5 | 25 | 3.00 |

According to Table B, in a reactor of suitable volume, the components were added in the following order of 1^{st} substrate (MA1 or MA2 obtained after step 7 of step a), 2^{nd} Glucose, 3^{rd} NADP, 4^{th} FAD, 5^{th} ET004 and 6^{th} PHBH. Reaction is carried out at 35° C./220 rpm for 98 hours. Samples were subdued to HPLC analysis for chromatographic profile comparison (samples were collected at time 0 h, 30 h, 96 h).

HPLC assay conditions:

(1) the mobile phase

Solution A: methanol;

Solution B: 1% phosphoric acid in ddH₂O;

A:B=30%:70%

(2) Column: Agilent C18, 250 mm*4.6 mm

(3) Detection wavelength: 210 nm

(4) Column temperature: 25° C.

(5) Flow Rate: 1 ml/min

Results:

By HPLC chromatography, an evolution of the peaks was observed through time with both mixtures MA1 and MA2 demonstrating further degradation of the mixtures during enzymatic treatment with a PHBH. In particular, the most interesting results were observed with enzymatic treatment of MA1 with M010-2 showing by HPLC a significant increase of peaks at 3.5 min and 4.2 min through time (see Tables 1 and 2)

TABLE 1
Resin R1

| | | | |
|---|---|---|---|
| | Peak area at 3.5 | Peak area at 4.1 | Peak area at 4.2 |
| Time (hrs) | min | min | min |

Strain M020-1 (ID-4)

| | | | |
|---|---|---|---|
| 0 | 103 | — | — |
| 30 | 206 | — | — |
| 96 | 958 | 937 | 150 |

Strain YM322-2 (ID-5)

| | | | |
|---|---|---|---|
| 0 | 330 | — | 372 |
| 96 | 489 | — | 575 |

Strain M010-2 (ID-6)

| | | | |
|---|---|---|---|
| 0 | 127 | — | 199 |
| 30 | 300 | — | 197 |
| 96 | 1011 | — | 1174 |

TABLE 2
Resin R2

| | | | |
|---|---|---|---|
| | Peak area at 3.5 | Peak area at 4.1 | Peak area at 4.2 |
| Time (hrs) | min | min | min |

None (ID-1)

| | | | |
|---|---|---|---|
| 0 | — | — | — |
| 30 | — | — | — |
| 96 | 182 | — | — |

Strain M012-2 (ID-2)

| | | | |
|---|---|---|---|
| 0 | 118 | — | — |
| 30 | 229 | — | — |
| 96 | 1071 | 2086 | — |

Strain YM322-2 (ID-3)

| | | | |
|---|---|---|---|
| 0 | 325 | — | — |
| 30 | 487 | — | — |
| 96 | 1449 | 1253 | 405 |

It was supposed that the mechanism of action of the para-hydroxybenzoate hydroxylase

(PHBH) was the hydroxylation of aryl rings. It was confirmed by running the following model reactions:

a. Model reaction 1—with 3,4-dihydroxybenzoic acid as substrate:

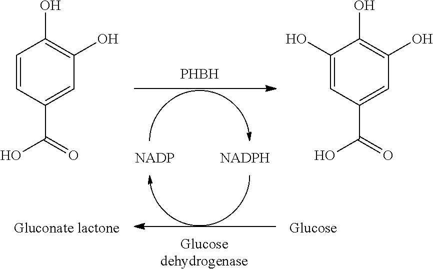

b. Model reaction 2—with Bisphenol A (BFA) as substrate:

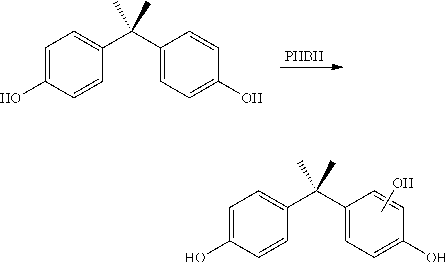

D—Enzymatic Treatment by Performing Concomitant Step b) and c)

Step a):

(1) Prepare a solution of 1.98 g Resin+50.65 g deionized H₂O; +90.00 g Na₂HPO₄.12H₂O, +189 g deionized H₂O are mixed at 60° C. until complete solubilization of the Na₂HPO₄.12H₂O salt;

(2) Evaporate the solution from the previous step at 80° C. under vacuum to remove the water, then add about 300 g of deionized H₂O, then evaporate again until complete removal of water,

(3) Repeat the 2nd step again;

(4) Add deionized H₂O at 90° C. to the dry solid obtained after step 3;

(5) Add 9.96 g β-Cyclodextrin to the mixture obtained after step 4 (total weight 142 g), and mix;

(6) Stir the mixture at 90° C. for two days;

(7) Dilute the mixture to obtain a volume of 200 ml;

The mixture obtained after performing step 1 to 7 on resin R1 is noted MA1.

The mixture obtained after performing step 1 to 7 on resin R2 is noted MA2.

Steps b) and c):

TABLE C

| | | Weight | | | | | | | | | |
|---|---|---|---|---|---|---|---|---|---|---|---|
| | | Of | | | | | | | | | |
| | | sub- | NaOH | Glu- | | | | NADP | FAD | | |
| | Sub- | strate | solution | cose | GSH | Glycine | ET004 | (20 g/l) | (4 g/l) | PHBH | GST |
| ID | strate | (g) | (ml) | (g) | (g) | (g) | (ml) | (ml) | (μl) | (ml) | (ml) |
| 1 | MA2 | 30 | 51.7 | 11.25 | 0.22 | 1.07 | 3.6 | 3 | 150 | 10 | 7 |
| 2 | MA1 | 30 | 50 | 11.25 | 0.22 | 1.07 | 3.6 | 3 | 150 | 9 | 7 |
| 3 | MA2 | 30 | 68.7 | 11.25 | 0.22 | 1.07 | 3 | 3 | 150 | 0 | 0 |
| 4 | MA1 | 30 | 68.7 | 11.25 | 0.22 | 1.07 | 3 | 3 | 150 | 0 | 0 |

According to table C, the compounds were added in a suitable reactor in the following order of: 1^{st} substrate (mixture MA1 or MA2), 2^{nd} aqueous solution of NaOH (pH9), 3^{rd} Glucose, 4^{th} reduced glutathione (GSH), 5^{th} Glycine, 6^{th} NADP, 7^{th} FAD, 8^{th} ET004, 9^{th} PHBH and 10^{th} GST. The reaction medium is stirred until reach homogeneity. The reaction is conducted at 30° C./220 rpm for 72-96 h. The reaction medium was sampled at 0 h, 20 h, 48 h, 72 h and 96 h. Control experiments were also conducted in which no enzyme was used.

The mixture obtained after performing step b) and c) on MA1 is noted MC1.

The mixture obtained after performing step b) and c) on MA2 is noted MC2.

The mixture obtained after conducting the experiment with no enzyme on MA1 is noted MC1-CTRL.

The mixture obtained after conducting the experiment with no enzyme on MA2 is noted MC2-CTRL.

HPLC assay condition

(1) the mobile phase

Solution A: methanol;

Solution B: 1% phosphoric acid in ddH2O;

A:B=30%:70%

(2) column: Agilent C18, 250 mm*4.6 mm

(3) Detection wavelength: 210 nm

(4) Column temperature: 25° C.

(5) Flow Rate: 1 ml/min

TABLE 3

| | | | |
|---|---|---|---|
| Time | Peak area at | Peak area at | Peak area at |
| (hrs) | 2.7 min | 3.5 min | 4.2 min |

MC1

| | | | |
|---|---|---|---|
| 0 | — | 101 | 0 |
| 20 | — | 262 | 108 |
| 72 | — | 1148 | 105 |
| 96 | — | 1927 | 158 |

MC1-CTRL

| | | | |
|---|---|---|---|
| 0 | — | — | — |
| 24 | — | 145 | — |
| 48 | — | 200 | — |
| 72 | — | 262 | — |

MC2

| | | | |
|---|---|---|---|
| 0 | 942 | 421 | — |
| 48 | 1339 | 606 | — |
| 72 | 1374 | 1174 | 385 |
| 96 | 1405 | 1241 | 283 |

MC2-CTRL

| | | | |
|---|---|---|---|
| 0 | — | — | — |
| 24 | — | 210 | — |
| 48 | — | 118 | — |
| 72 | — | 245 | — |

Table 3 above shows the influence of the presence of the enzymes GST and PHBH and their effect when used concomitantly. The evolution of the area of the peaks according to time shows an enrichment of molecular species that elude or coelude in specific retention times for mixtures MC1 and MC2 derived from resins R1 and R2 and treated with GST and PHBH. To the contrary, barely nothing happened with samples of resins R1 and R2 treated with no enzyme (MC1-CTRL and M2-CTRL).

E—Scale-Up

Step a)

1^{st} Step: Prepare a solution of 10.04 g Resin R1+102.08 g deionized H₂O; +100.01 g Na₂HPO₄.12H₂O, mix at 60° C. until complete solubilization of the Na₂HPO₄.12H₂O salt;

2^{nd} Step: Evaporate the solution from the previous step at 80° C. under vacuum to remove water, then add about 300 g of deionized H₂O, then evaporate again until complete removal of water;

3^{rd} Step: Repeat the 2nd step;

4^{th} Step: Add deionized water at 90° C. to the dry solid obtained after step 3;

5^{th} Step: Add 50.72 g 3-Cyclodextrin to the previous solution, and mix;

6^{th} Step: Stir the mixture at 90° C. for two days;

7^{th} Step: Dilute the mixture to obtain a volume of about 1200 ml in the reactor;

The broth obtained after the material dilution at the 7^{th} Step is actually used to explore the degradation of resin R2 and R2.

The mixture obtained after performing step 1 to 7 on resin R1 is noted MA1. The mixture obtained after performing step 1 to 7 on resin R2 is noted MA2. Steps b) and c)

TABLE D

| | | Weight of | Glu- | | | | NADP | FAD | | | NaOH |
|---|---|---|---|---|---|---|---|---|---|---|---|
| | | substrate | cose | GSH | Glycine | ET004 | (20 g/l) | (4 g/l) | PHBH | GST | 3M |
| N. | Substrate | (g) | (g) | (g) | (g) | (ml) | (ml) | (ml) | (ml) | (ml) | (ml) |
| 1 | MA1 | 1200 | 187 | 0.1 | 17.8 | 10 | 12.5 | 2.5 | 80 | 33 | 662 |
| 2 | MA2 | 1200 | 187 | 0.1 | 17.8 | 10 | 12.5 | 2.5 | 80 | 33 | 662 |

According to table D, the compounds were added in a 5 L reactor in the following order of: 1^{st} substrate (MA1 or MA2), 2^{nd} NaOH 3M, 3^{rd} Glucose, 4^{th} GSH, 5^{th} Glycine, 6^{th} NADP, 7^{th} FAD, 8^{th} ET004, 9^{th} PHBH and 10^{th} GST. The reaction medium is stirred at 200 rpm until homogeneity is reached. Compressed air was pumped at a rate of 1 L/min in the reactor. The reaction is conducted at 30° C./200 rpm for 66 h. The mixture obtained was filtrated and dried.

Starting from MA1, the dried mixture obtained is noted MC1.

Starting from MA2, the dried mixture obtained is noted MC2.

The mixture obtained after conducting the experiment with no enzyme on MA1 is noted MC1-CTRL.

The mixture obtained after conducting the experiment with no enzyme on MA2 is noted MC2-CTRL.

TABLE 4
Resin R1

| Time and temperature for | Weight MC1- | Weight |
|---|---|---|
| drying the resulting mixture | CTRL (g) | MC1 (g) |
| 0 hrs | 9.85 | 10.04 |
| 3 hrs at 90° C. | 9.77 | 7.98 |
| 5 hrs at 90° C. | 9.75 | 7.96 |

TABLE 5
Resin R2

| Time and temperature for | Weight MC2- | Weight |
|---|---|---|
| drying the resulting mixture | CTRL (g) | MC2 (g) |
| 0 hrs | 9.76 | 10.02 |
| 3 hrs at 90° C. | 9.69 | 8.64 |
| 5 hrs at 90° C. | 9.67 | 8.62 |

Tables 4 and 5 show that when both Resins R1 and R2 treated with GST and PHBH, a loss of material is observed.

Step d)

The dried mixtures MC1 and MC2 were sampled (around 1 g of mass) and treated with a 2M NaOH aqueous solution at 90° C. under stirring for 96 h (4 days). The mixtures were then filtrated, washed and dried at 90° C. for 5 hrs, and the loss of mass registered in Table 6 below.

Starting from MC1, the dried mixture obtained is noted MD1.

Starting from MC2, the dried mixture obtained is noted MD2.

The mixture obtained after conducting the experiment with no enzyme on MA1 is noted MD1-CTRL.

The mixture obtained after conducting the experiment with no enzyme on MA2 is noted MD2-CTRL.

TABLE 6

| | | mass after NaOH |
|---|---|---|
| | mass before | treatment and |
| | NaOH | drying at 90° C. |
| | treatment (g) | for 5 hrs (g) |
| MD1-CTRL | 1.01 | 1.0 |
| MD1 | 1.02 | 0.9 |
| MD2-CTRL | 1.00 | 0.96 |
| MD2 | 1.00 | 0.86 |

Resins R1 and R2 chemoenzymatically treated are more sensitive to the action of NaOH, losing higher amounts of mass, compared to untreated resins R1 and R2. The chemoenzymatic treatment was responsible for enhancing the solubility of portions of R1 and R2 in alkaline media.

F—Two Rounds of the Method According to the Invention

1^{st} Round:

step a)

(1) Prepare a solution of 1.98 g Resin R2+50.65 g ddH2O; +90.00 g Na₂HPO₄.12H₂O, +189 g ddH₂O, mix at 60° C. until complete solubilization of the Na₂HPO₄.12H₂O salt;

(2) evaporate at 80° C. under vacuum to remove the water, then add about 300 g of deionized water, then evaporate again until complete removal of water;

(3) Repeat the 2nd step;

(4) Add deionized water at 90° C. to the dry solid;

(5) Add 9.96 g β-Cyclodextrin to the previous solution (total weight 142 g), and mix;

(6) Stir the mixture at 90° C. for two days;

(7) Diluted the mixture to 200 ml;

(8) Resin R1 was dealt with the same process of (1)-(7).

The mixture obtained after performing step 1 to 7 on resin R1 is noted MA1. The mixture obtained after performing step 1 to 7 on resin R2 is noted MA2.

Steps b) and c)

TABLE E

| | | Weight | | | | | | | | | |
|---|---|---|---|---|---|---|---|---|---|---|---|
| | | of | NaOH | | | Gly- | | NADP | FAD | | |
| | Sub- | substrate | 3M | Glucose | GSH | cine | ET004 | (20 g/l) | (4 g/l) | PHBH | GST |
| ID | strate | (g) | (ml) | (g) | (g) | (g) | (ml) | (ml) | (μl) | (ml) | (ml) |
| 1 | MA2 | 30 | 51.7 | 11.25 | 0.22 | 1.07 | 3.6 | 3 | 150 | 10 | 7 |
| 2 | MA1 | 30 | 50 | 11.25 | 0.22 | 1.07 | 3.6 | 3 | 150 | 9 | 7 |

According to table E, the compounds were added in a suitable reactor in the following order of: 1^{st} substrate (MA1 or MA2), 2^{nd} NaOH 3M, 3^{rd} Glucose, 4^{th} GSH (substrate for GST enzyme), 5^{th} Glycine, 6^{th} NADP (cofactor), 7^{th} FAD (cofactor), 8^{th} ET004 (enzyme), 9^{th} PHBH (enzyme) and 10^{th} GST (enzyme). The reaction medium is stirred until reach homogeneity. The reaction is conducted at 30° C./220 rpm for 72-96 h.
(1) The resulting mixtures were left to decant and the supernatant was removed.
(2) Water was added, left to decant again, then the supernatant was removed.
(3) Step (2) was repeated until the supernatant was clear.
(4) The supernatant was removed.

Starting from MA1, the mixture obtained is noted MC1.

Starting from MA2, the mixture obtained is noted MC2.

Step d)

(1) Add 50 ml of 2 M NaOH solution to mixtures MC1 or MC2;

(2) Stir the mixture at 90° C. for 4 days;

(3) Remove the supernatant from the mixture and wash the solid three times with water.

Starting from MC1, the mixture obtained is noted MD1.

Starting from MC2, the mixture obtained is noted MD2.

Control experiments without enzymes PHBH and GST were also conducted.

2^{nd} Round:

Enzymatic Treatment in 2^{nd} Round (Steps b and c):

The solid from 1^{st} Round is mixed again with the other chemicals and enzymes in the following order: 1^{st} substrate (MD1 from 1^{st} Round), 2^{nd} NaOH 3M, 3^{rd} Glucose, 4^{th} GSH, 5^{th} Glycine, 6^{th} NADP, 7^{th} FAD, 8^{th} ET004, 9^{th} PHBH and 10^{th} GST. The reaction medium is stirred until reach homogeneity. The reaction is carried out at 30° C., 220 rpm for 120 h (5 days).

(1) The resulting mixture was left to decant and the supernatant was removed.

(2) Water was added, left to decant again, then the supernatant was removed.

(3) Step (2) was repeated until the supernatant was clear.

(4) The supernatant was removed.

Starting from MD1, the mixture obtained is noted MC1′.

NaOH Treatment in the 2^{nd} Round (Step d):

(1) Add 50 ml of 2 M NaOH solution to MC1′;

(2) Stir the mixture at 90° C. for 4 days;

(3) Remove the supernatant from the mixture and wash the solid three times with water;

(4) Dry the solid at 70° C.

Starting from MC1′, the mixture obtained is noted MD1′.

TABLE 7

| | Weight loss |
|---|---|
| Control R1 | 2% |
| R1-after 1^{st} round | 31% |
| R1-after 2^{nd} round | 53% |
| Control R2 | 4% |
| R2-after 1^{st} round | 24% |

1. A method for degrading an epoxy resin comprising the following successive steps:
a) adding in a solvent an epoxy resin R based on:
at least one aromatic compound R1 bearing at least two epoxide groups per molecule and comprising at least one aromatic ring bearing at least one glycidyloxy group, and
at least one curing agent R2,
to obtain mixture MA,
b) treating enzymatically the mixture MA obtained after step a), the treatment comprising a step of contacting the mixture MA with a glutathione S-transferase to obtain a mixture MB.

2. The method according to claim 1, further comprising a step c) performed after step b) or concomitantly with step b), step c) consisting in an enzymatic treatment which comprises a step of contacting mixture MA or mixture MB with a para-hydroxybenzoate hydroxylase to obtain a mixture MC.

3. The method according to claim 1, wherein enzymatic treatment consisting in step b) or step b) and step c) is followed by a successive step d) which consists in treating chemically mixture MB or mixture MC, the chemical treatment comprising a step of contacting mixture MB or mixture MC with an aqueous solution of a strong Bronsted base to obtain a mixture MD.

4. The method according to claim 1, wherein step a) comprises swelling of the epoxy resin R in the solvent.

5. The method according to claim 1, wherein in step a) the solvent is chosen among the group consisting in an aqueous solution of disodium phosphate and an aqueous solution of phosphoric acid and phenol.

6. The method according to claim 1, wherein in step b) the glutathione S-transferase is issued from a N. aromaticivorans strain.

7. The method according to claim 2, wherein in step c) the para-hydroxybenzoate hydroxylase is a mutated enzyme.

8. The method according to claim 3, wherein the aqueous solution of a strong Bronsted base in step d) is a sodium hydroxide aqueous solution.

9. The method according to claim 1, wherein the aromatic compound R1 corresponds to a diglycidyl ether of bisphenol.

10. The method according to claim 1, wherein the curing agent R2 is an amine or an imidazole derivative.

11. The method according to claim 3, wherein mixture MD obtained after step d) is then further submitted to steps b) to d) as defined above.

12. The method according to claim 3, wherein mixture MD obtained after step d) is further submitted to steps a) to d) as defined above.

13. (canceled)

14. (canceled)

15. A method for recycling a composite material CM comprising the following steps:
a′) providing a composite material comprising an epoxy resin R based on:
at least one aromatic compound R1 bearing at least two epoxide groups per molecule and comprising at least one aromatic ring bearing at least one glycidyloxy group, and
at least one curing agent R2,
b′) treating the composite material CM obtained after step a′), the treatment comprising a step of degrading the epoxy resin R with the method as defined in claim 1, in which case the epoxy resin R in step a) is the composite material CM,
c′) obtaining after step b′) the composite material with a reduced content in epoxy resin.

16. The method of claim 5, wherein in step a) the solvent is an aqueous solution of disodium phosphate.

17. The method of claim 2, wherein in step c) the para-hydroxybenzoate hydroxylase is a mutated enzyme presenting at least two point mutations L199V or L200V and Y385F.

18. The method of claim 9, wherein the aromatic compound R1 corresponds to a bisphenol A diglycidyl ether.
